(19) United States
(12) Patent Application Publication
(10) Pub. No.: US 2024/0182401 A1
(43) Pub. Date: Jun. 6, 2024

(54) A PROCESS FOR THE PREPARATION OF MIXED OXYBATE SALTS AND POLYMORPHS THEREOF

(71) Applicant: ALKEM LABORATORIES LIMITED, Mumbai (IN)

(72) Inventors: Jayprakash Ajitsingh PARIHAR, Mumbai (IN); Prashant Pandurang PAWAR, Mumbai (IN); Praveen Raosaheb SUPEKAR, Mumbai (IN); Ashutosh Akhilesh RAGHUWANSHI, Mumbai (IN); Vaibhav Vilas TATTE, Mumbai (IN)

(21) Appl. No.: 18/550,845

(22) PCT Filed: Mar. 19, 2022
(86) PCT No.: PCT/IN2022/050271

§ 371 Date: Sep. 15, 2023

(30) Foreign Application Priority Data
Mar. 20, 2021 (IN) 202121011961

(51) Int. Cl. C07C 51/41; A61K 31/19

(52) U.S. Cl. CPC C07C 51/412; A61K 31/19; C07B 2200/13

(57) ABSTRACT

The present invention provides an improved scalable process for preparation of mixed oxybate salts. More particularly, the present invention provides a process of preparation of a mixture of sodium, potassium, magnesium and calcium oxybate salts. Further, the present invention provides polymorphs of each of the individual salts and the mixed salt along with the methods of preparation of each.

### FIELD OF THE INVENTION

The present invention relates to an improved scalable process for the preparation of mixed oxybate. More particularly, the present invention relates to the process for the preparation of a mixture of sodium, potassium, magnesium and calcium oxybate salts. Further, the present invention relates to polymorphs of each of the individual salts and the mixed salt along with methods of preparation of each.

### BACKGROUND OF THE INVENTION

Mixed oxybate is also known as a mixture of calcium oxybate, magnesium oxybate, potassium oxybate, and sodium oxybate. Mixed oxybate is a central nervous system depressant indicated for the treatment of cataplexy or excessive daytime sleepiness (EDS). Chemically, the mixed oxybate is represented as a following structure:

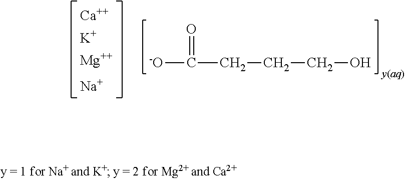

Calcium oxybate, magnesium oxybate, potassium oxybate, and sodium oxybate is approved in United States under the proprietary name XYWAV® as oral solution and marketed by Jazz Pharmaceuticals, Inc. The product XYWAVR contains 0.234 g calcium oxybate, Ca(C₄H₇O₃)₂; 0.096 g magnesium oxybate, Mg(C₄h₇O₃)₂; 0.13 g potassium oxybate, K(C₄H₇O₄); and 0.04 g sodium oxybate, Na(C₄H₇O₃) in dissociated form in the solution.

Oxybate also known as Gamma-hydroxybutyrate (GHB) is an endogenous compound with hypnotic properties found in many human body tissues. GHB is commonly encountered forensically as gamma-hydroxybutyric acid or in salt form as gamma-hydroxybutyrate typically as a sodium salt, also known as sodium oxybate. GHB is present, for example, in the mammalian brain and other tissues. In the brain, the highest GHB concentration is found in the hypothalamus and basal ganglia and GHB is postulated to function as a neurotransmitter. The neuropharmacologic effects of GHB include increases in brain acetylcholine, increases in brain dopamine, inhibition of GABA-ketoglutarate transaminase and depression of glucose utilization but not oxygen consumption in the brain. GHB treatment substantially reduces the signs and symptoms of narcolepsy, i.e., daytime sleepiness, cataplexy, sleep paralysis, and hypnagogic hallucinations. In addition, Oxybate increases total sleep time and REM sleep, and it decreases REM latency, reduces sleep apnea, and improves general anesthesia.

U.S. Pat. No. 9,555,017 discloses methods of making a mixture of salts of oxybate. The patent discloses a method of contacting gamma-butyrolactone (GBL) with two or more bases selected from the group consisting of sodium hydroxide (NaOH), potassium hydroxide (KOH), magnesium hydroxide (Mg(OH)₂), and calcium hydroxide (Ca(OH)₂), in a single reaction vessel under conditions sufficient to produce a mixture of two or more salts selected from the group consisting of a sodium salt of gamma-hydroxybutyrate (Na.GHB), a potassium salt of gamma-hydroxybutyrate (K.GHB), a magnesium salt of gamma-hydroxybutyrate (Mg.(GHB)₂), and a calcium salt of gamma-hydroxybutyrate (Ca.(GHB)₂)

Article by J. Ferris et al. (Forensic Science International 216 (2012) 158 162) teaches synthesis, characterization and detection of sodium, potassium, magnesium and calcium salt of oxybate. The article discloses that sodium, potassium, magnesium and calcium salts of gamma-hydroxybutyrate are synthesized from gamma-butyrolactone and the corresponding group 1 or 2 hydroxide.

U.S. Pat. No. 4,393,236 discloses a method for the production of magnesium and/or calcium salts of 4-hydroxybutyric acid by reacting a member of the groups consisting of 4-hydroxybutyric acid, 4-butyrolactone and mixtures thereof with a member of the group consisting of magnesium hydroxide, magnesium oxide, magnesium carbonate, calcium hydroxide, calcium oxide, calcium carbonate and mixtures thereof in an aqueous solution.

Thus, the disclosure of the available prior art fails to provide a simple and cost effective process of preparation of an oxybate salt. This necessitates for the preparation of an oxybate salt in its most effective form, especially a polymorph so as to exhibit significant therapeutic potential, the process of preparation being simple, cost effective and easily scalable for industrial application.

### OBJECTIVES OF THE INVENTION

The primary objective of the present invention is to provide a process of preparation of an oxybate salt.

Another objective of the present invention is to provide a process of preparation of a mixed oxybate salt.

Yet another objective of the present invention is to provide a scalable, cost effective and simple process of preparation of oxybate salts.

Yet another objective of the present invention is to provide polymorphs of mixed oxybate salts.

Yet another objective of the present invention is to provide a process of preparation of polymorphs of oxybate salts

### SUMMARY OF THE INVENTION

Accordingly, the present invention provides a scalable process for preparation of each of sodium oxybate, potassium oxybate, calcium oxybate, magnesium oxybate and a mixture of oxybate salts using a suitable solvent system. The present invention further provides different polymorphs of sodium oxybate, potassium oxybate, calcium oxybate, magnesium oxybate and mixed oxybate.

The present invention provides a process for the preparation of a mixed oxybate salt or a polymorph thereof, wherein the process includes steps of mixing at least one oxybate in a solvent and stirring at a temperature range from 0-30° C. to obtain a reaction mixture, wherein, at least one oxybate is selected from sodium oxybate, potassium oxybate, magnesium oxybate, or calcium oxybate. Then filtering the reaction mixture followed by washing with the solvent to obtain the mixed oxybate salt. The Mixed oxybate salt contains the sodium oxybate in an amount of 8±0.5% by wt., the potassium oxybate in an amount of 26±0.5% by wt., the magnesium oxybate in an amount of 19.2±0.5% by wt., and the calcium oxybate in an amount of 46.8±0.5% by wt.

Further, the present invention provides a mixed oxybate salt or a polymorph thereof comprising a sodium oxybate or a polymorph thereof present in an amount of 8±0.5% by wt., potassium oxybate or a polymorph thereof present in an amount of 26±0.5% by wt., magnesium oxybate or a polymorph thereof present in an amount of 19.2±0.5% by wt., and calcium oxybate or a polymorph thereof is present in an amount of 46.8±0.5% by wt.

Additionally, the present invention provides a mixed oxybate salt or a polymorph thereof comprising a sodium oxybate or a polymorph thereof, a potassium oxybate or a polymorph thereof, calcium oxybate or a polymorph thereof, and magnesium oxybate or polymorph thereof; obtained from the process as disclosed in the present invention using a critical solvent system, wherein the sodium oxybate is present in an amount of 8±0.5% by wt., the potassium oxybate is present in an amount of 26±0.5% by wt., the magnesium oxybate is present in an amount of 19.2±0.5% by wt., and the calcium oxybate is present in an amount of 46.8±0.5% by wt.

### BRIEF DESCRIPTION OF DRAWINGS OF THE INVENTION

FIG. 1 is a schematic representation of the process of preparation of magnesium oxybate and calcium oxybate salts.

FIG. 2 represents the XRD pattern of calcium oxybate salt, obtained by the procedure of Example 2.

FIG. 3 represents the XRD pattern of magnesium oxybate salt, obtained by the procedure of Example 4.

FIG. 4 represents the XRD pattern of potassium oxybate salt, obtained by the procedure of Example 5.

FIG. 5 represents the XRD pattern of sodium oxybate salt, obtained by the procedure of Example 1.

FIG. 6 represents the XRD pattern of mixed oxybate salt, obtained by the procedure of Example 6.

FIG. 7 represents the Differential Scanning calorimetry (DSC) of magnesium oxybate salt, obtained by the procedure of Example 4.

FIG. 8 represents the Differential Scanning calorimetry (DSC) of potassium oxybate salt, obtained by the procedure of Example 5.

FIG. 9 represents the Differential Scanning calorimetry (DSC) of calcium oxybate salt, obtained by the procedure of Example 2.

FIG. 10 represents the Thermogravimetric Analysis (TGA) of magnesium oxybate salt, obtained by the procedure of Example 4.

FIG. 11 represents the Thermogravimetric Analysis (TGA) of potassium oxybate salt, obtained by the procedure of Example 5.

FIG. 12 represents the Thermogravimetric Analysis (TGA) of calcium oxybate salt, obtained by the procedure of Example 2.

### DETAILED DESCRIPTION OF THE INVENTION

For the purpose of promoting an understanding of the principles of the present disclosure, reference will now be made to the embodiments illustrated in the drawings and specific language will be used to describe the same. It will nevertheless be understood that no limitation of the scope of the present disclosure is thereby intended, such alterations and further modifications in the illustrated composition, and such further applications of the principles of the present disclosure as illustrated therein being contemplated as would normally occur to one skilled in the art to which the present disclosure relates. The foregoing general description and the following detailed description are explanatory of the present disclosure and are not intended to be restrictive thereof. Unless otherwise defined, all technical and scientific terms used herein have the same meaning as commonly understood by one of ordinarily skilled in the art to which this present disclosure belongs. The process, and examples provided herein are illustrative only and not intended to be limiting.

The present invention provides a scalable process for the preparation of each of sodium oxybate, potassium oxybate, calcium oxybate, magnesium oxybate and mixture of oxybate salts using suitable solvent system. The present invention further provides different polymorphs of sodium oxybate, potassium oxybate, calcium oxybate, magnesium oxybate, and mixed oxybate.

The present invention provides a process of preparation of a mixed oxybate salt or a polymorph thereof, wherein the process includes steps of mixing at least one oxybate in a solvent and stirring at a temperature range from 0-30° C. to obtain a reaction mixture, wherein, at least one oxybate is selected from sodium oxybate, potassium oxybate, magnesium oxybate, or calcium oxybate. Then filtering the reaction mixture followed by washing with suitable solvent to obtain the mixed oxybate salt. Wherein, in the obtained mixed oxybate salt the sodium oxybate is present in an amount of 8±0.5% by wt., the potassium oxybate is present in an amount of 26±0.5% by wt., the magnesium oxybate is present in an amount of 19.2±0.5% by wt., and the calcium oxybate is present in an amount of 46.8±0.5% by wt.

The solvents include, but are not limited to, water, dichloromethane (CH2Cl2 or DCM), chloroform, tetrahydrofuran (THF), methyl-tetrahydrofuran, acetone, methyl ethyl ketone (MEK), methyl isobutyl ketone (MIBK), butanone, dimethylformamide (DMF), dimethylacetamide (DMAc), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, Nmethylformamide, acetonitrile (ACN or MeCN), dimethylsulfoxide (DMSO), 7 propionitrile, ethyl formate, methyl acetate (MeOAc), ethyl acetate (EtOAc), isopropyl acetate (IpOAc), butyl acetate (BuOAc), t-butyl acetate, hexachloroacetone, dioxane, sulfolane, N,N-dimethylpropionamide, nitromethane, nitrobenzene and hexamethylphosphoramide. Alcohols and glycols, methanol, ethanol, 1-propanol, 2-propanol, isopropanol (IPA), 1-butanol (1- BuOH), 2-butanol (2-BuOH), i-butyl alcohol, t-butyl alcohol, 2-nitroethanol, 2- fluoroethanol, 2,2,2-trifluoroethanol, ethylene glycol, 2-methoxyethanol, 2-ethoxyethanol, diethylene glycol, propylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol and methyl t-butyl ether (MTBE).

The solvents include, but are not limited to, water, ethanol, methanol, isopropyl alcohol, ethyl acetate, isopropyl acetate, isopropene acetate, butyl acetate, t-butyl acetate, cyclohexanone, heptane, hexane and the like. Preferably, the suitable solvents are selected from water, ethanol, cyclohexanone, propylene glycol, butanone, chloroform, ethyl acetate and mixture thereof.

In an embodiment, the present invention provides that the solvent in the disclosed process is selected from the group consisting of water, isopropyl acetate, n-heptane, toluene, n-butanol, t-butanol, or a mixture there of. Preferably, the solvent is a mixture of isopropyl acetate and n-heptane in the ratio of 1% to 99%

In an embodiment, the present invention provides that the solvent in the disclosed process is selected from the group consisting of water, isopropyl acetate, n-heptane, toluene, n-butanol, t-butanol, or a mixture there of. Preferably, the solvent is a water.

In an embodiment, the present invention provides a process of preparation of sodium oxybate or a polymorph thereof, the process includes the steps of reacting gamma butyrolactone with a base of sodium in a suitable solvent at a temperature ranging from 50-55° C. to obtain a reaction mass. Concentrating the reaction mass followed by cooling to a temperature ranging from 25-30° C. and adding a solvent to obtain a reaction mixture. Then filtering the reaction mixture followed by washing with a solvent and finally drying under vacuum.

In a preferred embodiment, the base of sodium is selected from sodium hydroxide, sodium t-butoxide or a combination thereof. More preferably, the base is sodium hydroxide.

In an embodiment, the present invention provides a process of preparation of potassium oxybate or a polymorph thereof, the process includes the steps of reacting gamma butyrolactone with a base of potassium in a suitable solvent at a temperature ranging from 50-55° C. to obtain a reaction mass. Concentrating the reaction mass followed by cooling to a temperature ranging from 25-30° C. and adding a solvent to obtain a reaction mixture. Then filtering the reaction mixture followed by washing with a solvent and finally drying under vacuum.

In a preferred embodiment, the base of potassium is selected from potassium hydroxide, potassium t-butoxide or a combination thereof. More preferably, the base is potassium hydroxide.

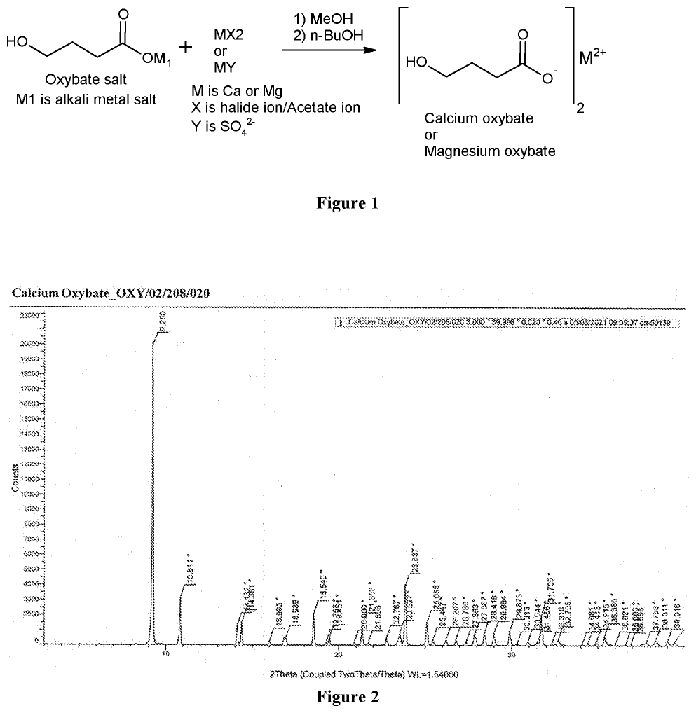

In an embodiment, the present invention provides a process of preparation of magnesium oxybate or a polymorph thereof, the process (as illustrated in FIG. 1) includes the steps of reacting a solution of sodium oxybate in a solvent with a solution of a magnesium salt in a solvent at a temperature ranging from 50-55° C. to obtain a reaction mixture. Filtering the reaction mixture under vacuum followed by addition of a solvent and cooling at a temperature ranging from 0-5° C.

In an embodiment, the present invention provides a process of preparation of magnesium oxybate or a polymorph thereof, the process includes the steps of mixing and reacting sodium oxybate and a magnesium salt in a solvent at a reaction temperature ranging from 20-40° C. to obtain a reactant mixture. Then stirring the reactant mixture at a temperature ranging from 30-40° C. for 30-45 minutes followed by cooling at a temperature ranging from 20-30° C. and filtering to obtain the filtrate. Raising the temperature of filtrate to a temperature ranging from 45-50° C. , adding the solvent and stirring for 4 hours followed by cooling at 25-30° C. , then stirring for 360 to 480 minutes, then adding solvent and filtering to separate a wet cake and a mother liquor. Mixing the wet cake with the solvent to obtain a reaction mixture, keeping the reaction mixture for 110-130 minutes at 45-50° C. , cooling the reaction mixture at 35-40° C. and stirring for 50-70 minutes. Adding the solvent and filtering the reaction mixture to obtain a wet cake and drying the wet cake by ramp wise temperature rising at 55-60° C. to obtain the magnesium oxybate or a polymorph thereof.

In a preferred embodiment, the magnesium salt is selected from the group consisting of magnesium chloride hexahydrate, magnesium sulphate, magnesium acetate or a combination thereof. More preferably, the magnesium salt is magnesium chloride hexahydrate.

In an embodiment, the present invention provides a process of preparation of calcium oxybate or a polymorph thereof, the process (as illustrated in FIG. 1) including the steps of reacting a solution of sodium oxybate in suitable solvent with a solution of a calcium salt in a solvent at a temperature ranging from 50-55° C. to obtain a reaction mixture. Filtering the reaction mixture under vacuum followed by addition of a suitable solvent and cooling at a temperature ranging from 0-5° C.

In a preferred embodiment, the calcium salt is selected from the group consisting of calcium chloride, calcium sulphate, calcium acetate, or a combination thereof. More preferably, the calcium salt is calcium chloride.

In an embodiment, the present invention provides that the suitable solvent used in the process of preparation of each of the individual salts is selected from the group consisting of methanol, n-butanol, t-butanol, acetonitrile, isopropyl alcohol, isopropyl acetate or a mixture there of. Preferably, the solvents are selected from methanol and n-butanol. More preferably, the solvents are selected from methanol and isopropyl alcohol.

Further, the present invention provides a mixed oxybate salt or a polymorph thereof comprising a sodium oxybate or a polymorph thereof present in an amount of 8±0.5% by wt., potassium oxybate or a polymorph thereof present in an amount of 26±0.5% by wt., magnesium oxybate or a polymorph thereof present in an amount of 19.2±0.5% by wt., and calcium oxybate or a polymorph thereof is present in an amount of 46.8±0.5% by wt.

Additionally, the present invention provides polymorph of mixed oxybate salt including a sodium oxybate or a polymorph thereof, a potassium oxybate or a polymorph thereof, calcium oxybate or a polymorph thereof, and magnesium oxybate or polymorph thereof. The mixed oxybate salt or a polymorph thereof is obtained from the process as disclosed in the present invention using a critical solvent system. Wherein, the sodium oxybate is present in an amount of 8±0.5% by wt., the potassium oxybate is present in an amount of 26±0.5% by wt., the magnesium oxybate is present in an amount of 19.2±0.5% by wt., and the calcium oxybate is present in an amount of 46.8±0.5% by wt.

Furthermore, the present invention provides a pharmaceutical composition comprising a mixed oxybate salt or a polymorph thereof, and pharmaceutically acceptable excipients. The said pharmaceutical composition is formulated into a suitable dosage form, preferably in a liquid dosage form.

Polymorphism, the occurrence of different crystal forms, is a property of some molecules and molecular complexes. A single oxybate compound, may give rise to a variety of polymorphs having distinct crystal structures and physical properties like melting point, thermal behaviors (e.g. measured by thermogravimetric analysis—“TGA”, or differential scanning calorimetry—“DSC”), X-ray powder diffraction (XRPD) pattern, infrared absorption fingerprint, Raman absorption fingerprint, and solid state (¹³C—) NMR spectrum. One or more of these techniques may be used to distinguish different polymorphic forms of a compound.

In another embodiment of the present invention, the sodium oxybate salt prepared by the process of the present invention is characterized by an XRPD pattern as depicted in FIG. 5.

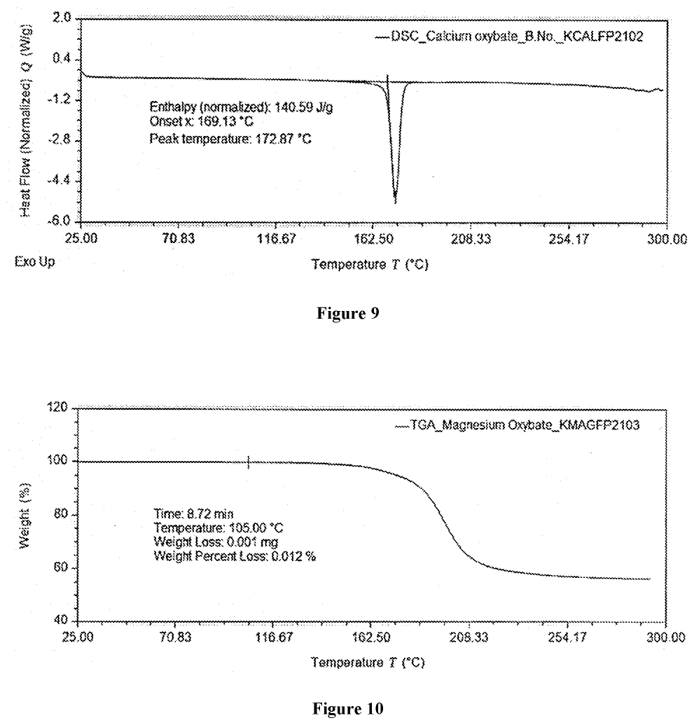

In another embodiment of the present invention, the calcium oxybate salt prepared by the process of the present invention is characterized by an XRPD pattern as depicted in FIG. 2, or a DSC as depicted in FIG. 9, or a TGA as depicted in FIG. 12.

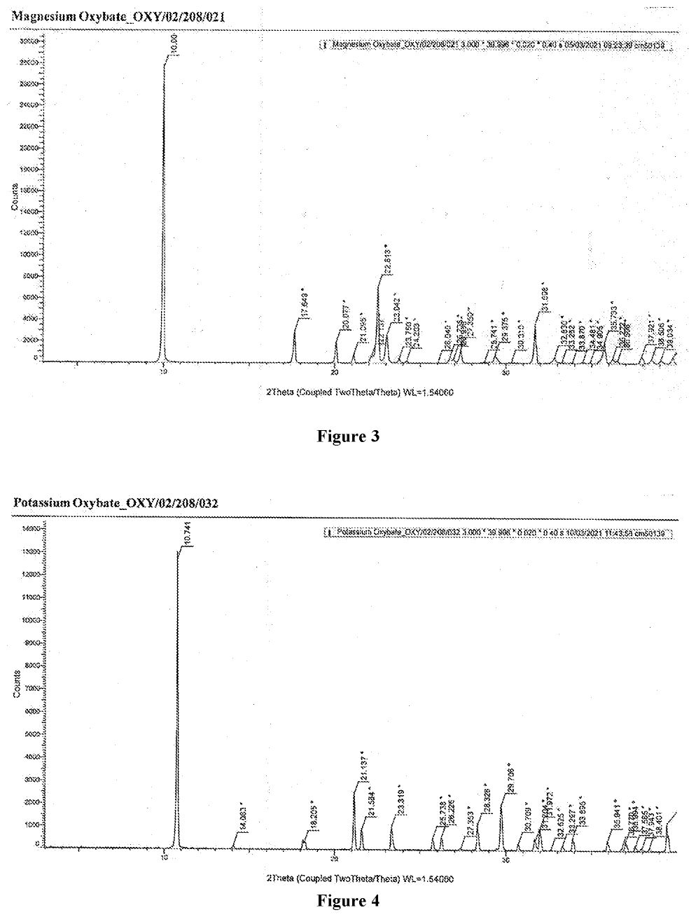

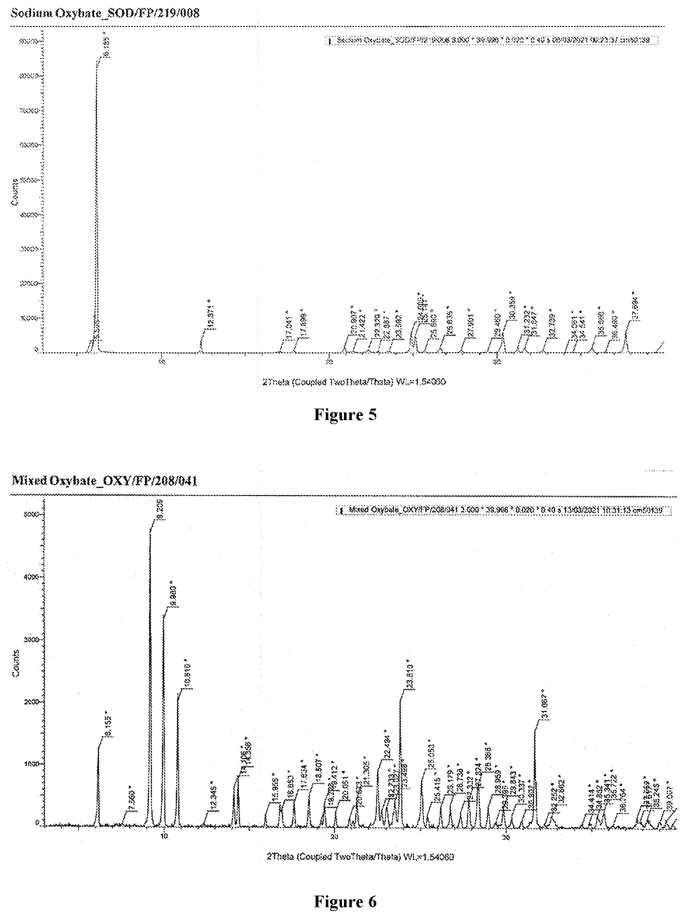

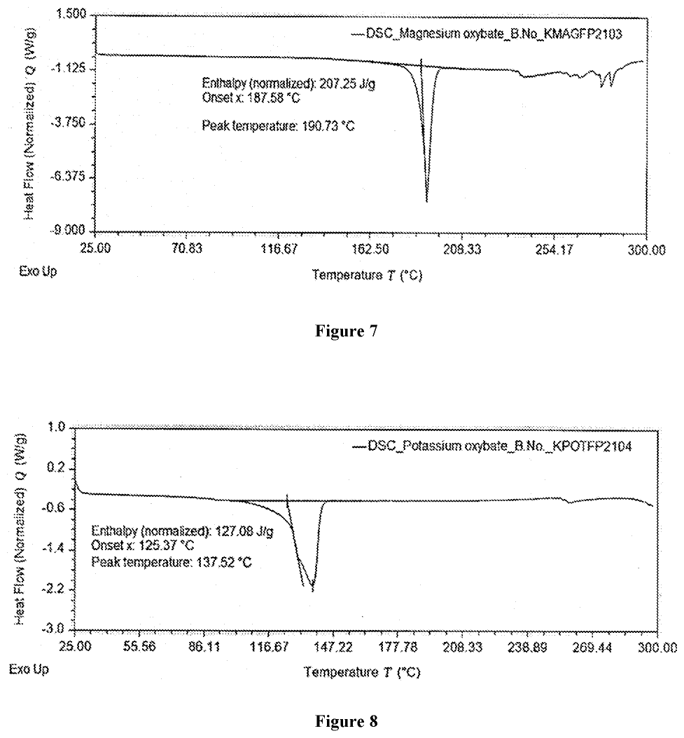

In another embodiment of the present invention, the magnesium oxybate salt prepared by the process of the present invention is characterized by an XRPD pattern as depicted in FIG. 3, or a DSC as depicted in FIG. 7, or a TGA as depicted in FIG. 10.

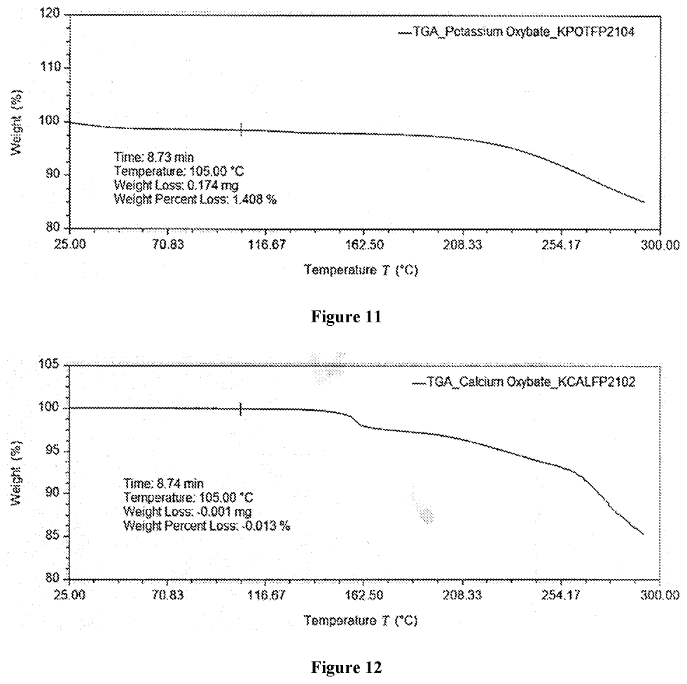

In another embodiment of the present invention, the potassium oxybate salt prepared by the process of the present invention is characterized by an XRPD pattern as depicted in FIG. 4, or a DSC as depicted in FIG. 8, or a TGA as depicted in FIG. 11. In an embodiment, the present invention provides that the mixed oxybate salt or a polymorph thereof as and when used for cataplexy or excessive daytime sleepiness.

The invention is further exemplified by the following non-limiting examples, which are illustrative representing the preferred modes of carrying out the invention. The invention scope is not limited to these specific embodiments only but should be read in conjunction with what is disclosed anywhere else in the specification together with the general understanding of a person skilled in the art.

### EXAMPLES

### Example 1

### Process for Preparation of Sodium Salt

To a stirred solution of gamma butyrolactone (50 g, 1 mole eq.) in ethanol (250 mL) was added sodium hydroxide (32.59 g, 1 mol eq) and the resultant solution was stirred further at 50-55° C. for 3 hours, cooled to 25-30° C. Solid was filtered and washed with ethanol (25 mL) and dried under vacuum. Output: 54 g; yield: 89%; HPLC purity: 99.87%.

### Example 2

### Process for Preparation of Calcium Salt

To a solution of Sodium oxybate (10 gm, 1 mole eq.) in methanol (40 mL) calcium chloride (4.23 gm, 0.50 mole eq) in methanol (25 ml) was added and heated to 50-55° C. for 2 hours to obtain a reaction mixture. Reaction mixture was filtered under vacuum. The filtrate was concentrated up to 1 volume, 4 volume of n-butanol was added and cooled to 0-5° C. Solid was filtered and washed with n-butanol (10 ml, 1 V) and dried under vacuum. The experimental details related to the preparation of the calcium salt is summarized in Table 1.

Output: 8.3 g; yield: 84.95%; HPLC purity: 99.66%

### Example 3

### Process for Preparation of Calcium Salt

To a solution of Sodium oxybate (100 gm, 1 mole eq.) in methanol (400 mL,4V) calcium chloride (44 gm, 0.50 mole eq) in methanol (200 mL, 2V) was added and heated to 50-55° C. for 2 hours to obtain a reaction mixture. Reaction mixture was filtered under vacuum. The filtrate was concentrated up to 2 volume at temperature 50-55° C. added methanol (500 mL, 5V) and cooled to 0-30° C. Solid was filtered and washed with methanol (100 ml, 1 V) and dried under vacuum. The experimental details related to the preparation of the calcium salt is summarized in Table 1.

Output: 75.6 g; yield: 77.14%.

### Example 4

### Process for Preparation of Magnesium Salt

To a solution of Sodium oxybate (100 gm, 1 mole eq.) in methanol (250 mL) magnesium chloride hexahydrate (37.7 gm, 0.50 mole eq) in methanol (50 mL) was added and heated to 50-55° C. for 3 hours to obtain a reaction mixture. Reaction mixture was filtered under vacuum. The filtrate was concentrated up to 1 volume, n-butanol (100 mL) was added and cooled to 0-5° C. Solid was filtered and washed with n-butanol (25 mL, 1 V) and dried under vacuum. The experimental details related to the preparation of the calcium salt is summarized in Table 2.

Output: 23.27 gm; yield: 87.59%; HPLC purity: 99.78%.

### Example 4a

### Process for Preparation of Magnesium Salt

Preparing a reaction solution by adding Sodium oxybate (100 gm, 1 mole eq.) in methanol (250 mL) then stirring under nitrogen for 10-15 minutes followed by cooling at 20-25° C. , then adding magnesium chloride anhydrous (37.76 g, 0.50 mole eq), followed by stirring for 30-45 minutes at 25-35° C. and then cooling at 20-30° C. under nitrogen atmosphere. Filtering the obtained reaction solution to separate the wet cake and the filtrate, then washing the wet cake with 50 ml mixture of methanol and Isopropyl alcohol (1:1 or 25ml: 25ml). The filtrate was concentrated at 45-50° C. and 1800 ml of Isopropyl alcohol is added to obtain the reaction mass, stirring the reaction mass for 230-250 minutes at 40-50° C. , followed by cooling at 25-35° C. and then again stirring the reaction

1. A process for preparing a mixed oxybate salt or a polymorph thereof, wherein the process comprises:
(i). mixing at least one oxybate in a solvent and stirring at a temperature range from 0-30° C. to obtain a reaction mixture, wherein, at least one oxybate is selected from sodium oxybate, potassium oxybate, magnesium oxybate, or calcium oxybate;
(ii). filtering the reaction mixture followed by washing with the solvent to obtain the mixed oxybate salt or the polymorph thereof; and
wherein, the mixed oxybate salt or the polymorph thereof comprises at least one or a combination of a sodium oxybate or a polymorph thereof present in an amount of 8±0.5% by wt., potassium oxybate or a polymorph thereof present in an amount of 26±0.5% by wt., magnesium oxybate or a polymorph thereof present in an amount of 19.2±0.5% by wt., or calcium oxybate or a polymorph thereof is present in an amount of 46.8±0.5% by wt.

2. The process as claimed in claim 1, wherein, the solvent is selected from the group consisting of water, dichloromethane (CH₂Cl₂ or DCM), chloroform, tetrahydrofuran (THF), methyl-tetrahydrofuran, acetone, methyl ethyl ketone (MEK), methyl isobutyl ketone (MIBK), butanone, dimethylformamide (DMF), dimethylacetamide (DMAc), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, Nmethylformamide, acetonitrile (ACN or MeCN), dimethylsulfoxide (DMSO), 7 propionitrile, ethyl formate, methyl acetate (MeOAc), ethyl acetate (EtOAc), isopropyl acetate (IpOAc), butyl acetate (BuOAc), t-butyl acetate, hexachloroacetone, dioxane, sulfolane, N,N-dimethylpropionamide, nitromethane, nitrobenzene and hexamethylphosphoramide, Alcohols and glycols, methanol, ethanol, 1-propanol, 2-propanol, isopropanol (IPA), 1-butanol (1-BuOH), 2-butanol (2-BuOH), i-butyl alcohol, t-butyl alcohol, 2-nitroethanol, 2-fluoroethanol, 2,2,2-trifluoroethanol, ethylene glycol, 2-methoxyethanol, 2-ethoxyethanol, diethylene glycol, propylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol and methyl t-butyl ether (MTBE).

3. The process as claimed in claim 2, wherein, the solvent is selected from the group consisting of water, ethanol, methanol, isopropyl alcohol, ethyl acetate, isopropyl acetate, isopropene acetate, butyl acetate, t-butyl acetate, cyclohexanone, heptane, hexane and a combination thereof.

4. The process as claimed in claim 2, wherein, the solvent is selected from the group consisting of water, ethanol, cyclohexanone, propylene glycol, butanone, chloroform, ethyl acetate, and a combination thereof.

5. The process as claimed in claim 2, wherein, the solvent is selected from the group consisting of water, isopropyl acetate, n-heptane, toluene, n-butanol, t-butanol, methanol and a combination thereof.

6. The process as claimed in claim 2, wherein, the solvent is selected from a mixture of isopropyl acetate and n-heptane, wherein, the isopropyl acetate is 1% and n-heptane is 99%.

7. The process as claimed in claim 2, wherein, the solvent is water, preferably purified water.

8. The process as claimed in claim 1, wherein, the sodium oxybate or the polymorph thereof is prepared by a process comprising the steps of:
(i). reacting a gamma butyrolactone with a base of sodium in a solvent at a temperature ranging from 50-55° C. to obtain a reaction mass;
(ii). concentrating the reaction mass followed by cooling at a temperature ranging from 25-30° C. and adding the solvent to obtain a reaction mixture; and
(iii). filtering the reaction mixture followed by washing with the solvent and drying under vacuum.

9. The process as claimed in claim 8, wherein, the base of sodium is selected from sodium hydroxide, sodium t-butoxide, and a combination thereof.

10. The process as claimed in claim 8, wherein, the base of sodium is sodium hydroxide.

11. The process as claimed in claim 1, wherein, the potassium oxybate or a polymorph thereof is prepared by a process comprising the steps of:
(i). reacting a gamma butyrolactone with a base of potassium in a solvent at a temperature ranging from 50-55° C. to obtain a reaction mixture, wherein, the base of potassium is selected from potassium hydroxide, potassium t-butoxide, and a combination thereof;
(ii). concentrating the reaction mixture followed by cooling at a temperature ranging from 25-30° ° C. and adding the solvent to obtain a reaction mixture;
(iii). filtering the reaction mixture followed by washing with the solvent and drying under vacuum.

12. (canceled)

13. The process as claimed in claims 11, wherein, the base is potassium hydroxide.

14. The process as claimed in claim 1, wherein, the magnesium oxybate or a polymorph thereof is prepared by a process comprising the steps of:
(i). mixing and reacting sodium oxybate and a magnesium salt in a solvent at a reaction temperature ranging from 20-40° C. to obtain a reaction mixture, wherein, the magnesium salt is selected from the group consisting of magnesium chloride hexahydrate, magnesium sulphate, magnesium acetate, and a combination thereof;
(ii). stirring the reaction mixture at a temperature ranging from 30-40° ° C. for 30-45 minutes followed by cooling at a temperature ranging from 20-30° C. and filtering to obtain the filtrate;
(iii). raising the temperature of filtrate to a temperature ranging from 45-50° C. , adding the solvent and stirring for 4 hours followed by cooling at 25-30° C. , then stirring for 360 to 480 minutes, then adding solvent and filtering to separate a wet cake and a mother liquor;
(iv). mixing the wet cake with the solvent to obtain a reaction mixture, keeping the reaction mixture for 110-130 minutes at 45-50° C. , cooling the reaction mixture at 35-40° C. and stirring for 50-70 minutes; and
(v). adding the solvent and filtering the reaction mixture to obtain a wet cake and drying the wet cake by ramp wise temperature rising at 55-60° C. to obtain the magnesium oxybate or a polymorph thereof.

15. (canceled)

16. The process as claimed in claim 14, wherein, the magnesium salt is magnesium chloride hexahydrate.

17. The process as claimed in claim 1, wherein, the calcium oxybate or a polymorph thereof is prepared by a process comprising the steps of:
(i). reacting a solution of sodium oxybate with a calcium salt in a solvent at a temperature ranging from 50-55° C. to obtain a reaction mixture, wherein, the calcium salt is selected from the group consisting of calcium chloride, calcium sulphate, calcium acetate and a combination thereof; and
(ii). filtering the reaction mixture under vacuum followed by addition of a suitable solvent and cooling at a temperature ranging from 0-5° C.

18. (canceled)

19. The process as claimed in claim 17, wherein, the calcium salt is calcium chloride.

20. The process as claimed in claim 8, wherein, the solvent is selected from the group consisting of methanol, n-butanol, t-butanol, acetonitrile, isopropyl acetate, and a combination there of.

21. The process as claimed in claim 19, wherein, the solvent is selected from methanol, n-butanol, and a combination thereof.

22. A pharmaceutical composition comprising a mixed oxybate salt or a polymorph thereof as prepared by the process claimed in claim 1, and pharmaceutically acceptable excipients.

23. The pharmaceutical composition as claimed in claim 22, wherein the said pharmaceutical composition is formulated into a liquid dosage form.
